(19) United States
(12) Patent Application Publication
(10) Pub. No.: US 2026/0232856 A1
(43) Pub. Date: Aug. 13, 2026

(54) METHOD FOR RECOVERY OF MATERIAL FROM POST-USE ABSORBENT SANITARY PRODUCTS

(71) Applicant: I-FORIA ITALIA S.R.L., Pescara (IT)

(72) Inventors: Marcello SOMMA, Pescara (IT); Tonino CARUSO, Salerno (IT); Giuseppe LANDOLFO, Pescara (IT)

(73) Assignee: I-FORIA ITALIA S.R.L., Pescara (IT)

(21) Appl. No.: 19/470,867

(22) PCT Filed: Apr. 15, 2024
(86) PCT No.: PCT/IB2024/053681

§ 371 Date: Sep. 30, 2025

(30) Foreign Application Priority Data
Apr. 14, 2023 (IT) 102023000007323

(51) Int. Cl. A61L 2/18; A61L 101/02; A61L 103/15; B01J 20/24

(52) U.S. Cl. CPC A61L 2/18; B01J 20/24; A61L 2101/02; A61L 2103/15

(57) ABSTRACT

A method to sterilize and decontaminate after use absorbent sanitary products contaminated with organic compounds deriving from body metabolism including also drug residues, said absorbent sanitary products, including plastic fractions and at least one between super-absorbent polymers (SAP) and cellulose, includes the steps of: —grinding at least once the absorbent sanitary products preferably to homogeneous size equal to or lower than 30 cm; —sterilizing the absorbent sanitary products by heating at a temperature equal to or lower than 140° C. and a relative pressure between 1 bar and 3 bars; —oxidizing sanitary products with hypochlorite in aqueous solution at a temperature lower than 80° C.

### CROSS REFERENCE TO THE RELATED APPLICATIONS

This application is the national phase entry of International Application No. PCT/IB2024/053681, filed on Apr. 15, 2024, which is based upon and claims priority to Italian Patent Application No. 102023000007323, filed on Apr. 14, 2023, the entire contents of which are incorporated herein by reference.

### TECHNICAL FIELD

The present invention refers to a method for the recovery of material from absorbent sanitary products, more particularly for the recovery of material from post-use absorbent sanitary products contaminated with organic compounds deriving from both human and animal body metabolism e.g. pets like cats.

### BACKGROUND

The absorbent sanitary products intended for human and animal use are generally composed of different materials including plastic, cellulose and super-absorbent polymers (SAP). These materials have economic value even after usage of the health product; therefore, their recovery and reuse become important.

However, the recovery of these materials from post-use absorbent sanitary products, before they can be traded again, passes through the overcoming of a series of critical issues due to the presence of organic contaminants deriving from body metabolism.

A first criticality is represented by the achievement of suitable chemical-physical characteristics, to be obtained by means of decontamination processes, which allow subjects deriving from these post-use absorbent sanitary products to be no longer qualified as waste but as regenerated materials (or secondary raw materials), for instance to create other absorbent products. The achievement of a level of decontamination necessary to ensure that these subjects are no longer considered as waste is a well-known process said oxidation. The use of chemical oxidizers is equally known, such as hydrogen peroxide, persulfates and ozone that have a standard potential value E° expressed in Volt greater than 1.8.

Numerous decontamination treatments are known in the art however they appear expensive for the type of oxidizers used. In fact, due to their aggressive action, these oxidizers guarantee the absence of risks in the reuse of the decontaminated material but at the same time negatively affect the yield and quality of the recycled materials.

Document WO2022137008 A1 describes, for instance, a sterilization-oxidation process of post-use absorbent sanitary products using a gas (i.e. ozone) as an oxidizing agent at temperatures higher or equal to 60° C.

Document US2019169795 A1 describes a method for the recovery of cellulose fiber from absorbent articles. In particular, the process provides for the separation of SAP from cellulose by means of oxidative depolymerization and consequent solubilization of the super absorbent polymer (in the form of LPA) obtained by soaking these articles in a sodium hypochlorite solution.

In WO2021130575 A1 a process is described for the recycling of absorbent sanitary articles similar to the previous one but wherein the depolymerization-solubilization of SAP in LPA takes place through the use of more aggressive oxidizers (e.g. Persulfates).

The WO2014203922 A1 document describes a process of sterilization-whitening of sanitary products through the use of sodium hypochlorite in a pH range between neutrality and weak acidity.

In addition, JP2002292304 document describes a process to disinfect diapers using sodium hypochlorite in process conditions that are not defined and wherein hypochlorite is used exclusively as disinfectant.

The processes described in known art do not allow efficient recovery in terms of quality and quantity of the materials used for the realization of the absorbent sanitary products.

### SUMMARY

The main purpose of the present invention is to provide a process for the recycling of post-use absorbent sanitary products, which allows to overcome the critical issues and limits that emerged in known art, which does not affect or relieve the quality of the treated materials and allows efficient recovery.

Another purpose of the present invention is to provide a process that provides for the use of an oxidizer of easy availability and reduced aggressiveness compared to other oxidizers used in known art, which prevent the degradation of the recycled material, and which does not affect the quality of the recovered products.

A further purpose is the neutralization of the oxidizing agent using a deactivating agent, typically a reducer possibly combined with an acidifying agent, easy to find and use.

Additional purpose of the present invention is to provide a process that includes the oxidation, neutralization and sterilization steps of these post-use absorbent sanitary products for their decontamination, sanitization and sterilization.

A still further purpose is to make available intermediate products and final recycled products obtained from the decontaminating, sanitizing and sterilizing treatment on post-use absorbent sanitary products.

These and further purposes can be achieved through a method to sterilize and decontaminate after use absorbent sanitary products contaminated with organic compounds deriving from body metabolism including drug residues. These absorbent sanitary products include plastic fractions, and at least one between super-absorbent polymers (SAP) and cellulose.

The invention method includes at least the steps of:
grinding post-use absorbent sanitary products preferably to a homogeneous average size equal to or lower than 40 cm.
oxidizing sanitary products with hypochlorite in aqueous solution at a temperature lower than 80° C.;
sterilizing the absorbent sanitary products by heating at a temperature equal to or lower than 140° C. and relative pressure between 1 bar and 3 bars.

In this way components such as plastic, super-absorbent polymers (SAP) and possibly cellulose contained in post-use absorbent sanitary products are sufficiently decontaminated both from organic compounds deriving from body metabolism and unwanted chemical residues of drugs, adopted both for the human beings and for animals, in particular pets.

These absorbent products can include plastic, and at least one between super-absorbent and cellulose polymers.

According to a preferred embodiment, the step of grinding produces an average homogeneous size equal to or lower than 40 cm, preferably 30, preferably equal to or lower than 10 cm.

Preferably, the method further includes the inactivation step of these chemical residues eventually still present on post-use absorbent sanitary products remained at the end of the oxidation step through the use of an antioxidant, in particular selected in the group including carboxylic organic acids or hydrogen peroxide when the oxidizer previously used was stabilized through hydroxides and/or carbonates.

Other advantages of the present invention are discussed in the description and mentioned in dependent claims.

### BRIEF DESCRIPTION OF THE DRAWINGS

The invention is further described on the basis of examples not limited to the relative scope and illustrated by way of example in the following figures, which refer respectively to:

FIG. 1. Oxidation in reactor. The oxidation and oxidizing inactivation steps take place in sequence within the reactor. The arrows indicate a handling of the processed material, from one component of the system to another.

FIG. 2. Oxidation in autoclave. The oxidation step takes place in the autoclave immediately before sterilization. The oxidizing inactivation step takes place at the same time as cooling, always within the autoclave.

FIG. 3. Post sterilization in autoclave oxidation. Oxidation takes place in the autoclave, after the cooling step. The inactivation step follows oxidation, always in the autoclave. The arrows indicate a handling of the processed material, from one component of the system to another.

FIG. 4. Autoclave-dried mixed fraction composed of cellulose, SAP and polyolefins. Absence of bleaching.

FIG. 5. Autoclave-dried mixed fraction composed of cellulose, SAP and polyolefins. Evidence of post-treatment bleaching with sodium hypochlorite, according to the present invention.

FIG. 6. Fraction of only post-treatment cellulose separated from the mix out of the autoclave. Evidence of post-treatment bleaching with sodium hypochlorite, according to the present invention.

FIG. 7. ATR spectrum (Attenuated Total Reflectance), of pure SAP, available on the market. The 3000 cm⁻¹ band is due to water and is not characteristic of the product under examination.

FIG. 8. ATR spectrum (Attenuated Total Reflectance) of post-use SAP, obtained as indicated in example 4. The band at 3000 cm⁻¹ is due to water and is not characteristic of the product under examination.

FIG. 9. ATR spectrum (Attenuated Total Reflectance) of the mixed polyolefin post-use fraction separated from cellulose and SAP. The main bands are consistent with those known shown for polypropylene (FIG. 10).

FIG. 10. ATR spectrum (Attenuated Total Reflectance) of pure polypropylene (data available on the SpectraBase® database of John Wiley & Sons, Inc., spectrabase.com/).

FIG. 11. At the top, ATR spectrum (Attenuated Total Reflectance) relating to the fraction of cellulose recovered after thermo-oxidative post-treatment. Below, a well-known spectrum of the pure cellulose, available on the SpectraBase® database of the John Wiley & Sons, Inc., spectrabase.com/).

FIG. 12. ATR spectrum (Attenuated Total Reflectance) of pure linear polyacrylate, LPA, available on the market, 40% in water. The 3000 cm-1 band is due to water and is not characteristic of the product under examination.

FIG. 13. ATR spectrum (Attenuated Total Reflectance) of the soluble compound extracted in water obtained by oxidation of the SAP in the conditions of the known art (US 2022/0257823). The spectrum is compatible with that of pure LPA (FIG. 12), apart from the band at 1000-1100 cm⁻¹ relating to the sulphates deriving from Caroat®, that of CO₂ around 2350 cm⁻¹, and the one at 3000 cm⁻¹, due to water and not characteristic of the product under examination.

FIG. 14. ¹³C-NMR spectrum of linear polyacrylate, LPA, soluble in water and obtained from release tests carried out downstream of the oxidative dereticulation of the SAP superabsorbent polymer (Example 5). The signal of the methylenic —CH₂— at about 84 ppm typical of the cross-linkers of the SAP cross linked polymer (FIG. 16) is absent.

FIG. 15. ¹³C-NMR spectrum of pure linear polyacrylate, LPA, soluble in water shown in the literature, ref 1. The signal of the methylenic —CH₂— at about 84 ppm typical of the cross-linkers of the SAP cross linked polymer is absent (FIG. 16).

FIG. 16. SAP ¹³C-NMR spectrum, pure, insoluble in water, reported in literature, Ref. 1. The signal of the methylenic —CH₂— to about 84 ppm typical of the cross-linkers of the cross-linked SAP polymer is present.

### DEFINITIONS

in the present document, the wording “liquid/solid ratio” means a volume/mass ratio wherein the volume, expressed in liters [L], is that of the liquid used, typically water or aqueous solution (e.g. oxidizer aqueous solution) used, while the mass, expressed in kilograms [kg], is that of the sanitary products to be treated.
in the present document, with the wording “heated autoclave”, we mean an autoclave with a shell wherein a hot fluid flows which, by heating the body of the autoclave, indirectly heats the material contained inside.
in the present document, with the wording “thermal sterilization cycle” we mean a sequence of exposure to different temperatures and pressures, for certain time intervals, of the material to be sterilized, in order to sterilize the material
In the present document, for “post-use absorbent sanitary products” we mean for instance, but not limited to: diapers for babies, adult diapers, absorbent for incontinence for adults, hygienic absorbents, bed traverses, sheets, covers, absorbent material for cat litter boxes such as traverses and hygienic bags for animals, toilet paper, intimate hygiene wipes, napkins etc.
In the present document, with the wording “traverse for animals” we mean a pre-assembled element, including a layer of absorbent or super absorbent material between two plastic films, which can be placed, for instance, inside a litter.
in the present document, with the wording “Hygienic bag for animals” we mean a package containing super absorbent material in flakes to be poured into a litter in a similar way to what would be done with the animal sand.
In the present document, post-use absorbent sanitary products can optionally contain no cellulose. Said cellulose-free products can be subjected to the same process conditions as the present invention. An example of said sanitary products substantially cellulose-free is the Pampers Progressi® line.
In the present document, the term “m/v” indicates a mass/volume ratio, wherein the mass is expressed in kilograms [kg] and the volume in liters [L].
In the present document, the “antioxidant” and “reducing” words are synonymous.
in the present document, the words “approximately” or “around” as used here when referring to a measurable value such as a quantity, a temporal duration, and the like, are intended to enclose variations of ±20%, ±10%, ±5%, ±1%, or ±0.1% with a specified value, where these variations are appropriate to perform the described methods.
in the present document, “release test” means a simulated test of contaminants release carried out by placing in contact, for a defined time (contact time), a known quantity of a solid with a well-known volume of an extracting agent (bleaching); and then separating the two steps to obtain a liquid eluate in the most appropriate solvent, as per method UNI EN 12457-2: 2004.

### DETAILED DESCRIPTION OF THE EMBODIMENTS

In the present invention, the inventors identified a procedure for the decontamination and sterilization of post-use absorbent sanitary materials wherein said absorbent materials include, in addition to the biological waste of human and animal body metabolism, also traces of metabolized drugs.

This process provides for the use of economic and easily available oxidizers that allow to recover a quantity of super-absorbent polymers (SAP). This advantage becomes reachable thanks to a not very aggressive oxidizing action that is performed in the decontamination step and which allows to avoid damage to the treated materials for recycling, such as cellulose, plastic and super-absorbent polymers (SAP).

Not limiting examples of drugs or compounds contained in drugs and absorbed in post-use absorbent sanitary materials according to the invention are:

Chemical compound

| | |
|---|---|
| 1 | Clavulanic acid |
| 2 | Amoxicillin |
| 3 | Ampicillin |
| 4 | Cefaclor (as cephalosporin) |
| 5 | Clarithromycin |
| 6 | Ciprofloxacin |
| 7 | Levofloxacin |
| 8 | Azithromycin |
| 9 | Hydrocortisone acetate |
| 10 | Hydrochlorothiazide (as benzothiadiazina) |
| 11 | Diazepam (as benzodiazepin) |
| 12 | Ibuprofen |
| 13 | Diclofenac |
| 14 | Acetylsalicylic acid |
| 15 | Bicalutamide |
| 16 | Metformin |
| 17 | Pioglitazone |
| 18 | Clopidogrel |
| 19 | Lansoprazol |
| 20 | Telmisartan |
| 21 | Torasemid |
| 22 | Lovastatin |
| 23 | Atorvastatin |
| 24 | Clodronic acid |

With aggressive decontamination, we mean the use of chemical oxidizers having a standard potential E° greater than 1.8 V and therefore capable of giving rise to strong thermodynamic reactions, capable of reducing the contaminants present in post-use absorbent sanitary products.

Furthermore, it is necessary to keep in mind that the reactivity of the chemical agents is not only function of the intrinsic characteristics of the species under examination but also of other parameters such as temperature, time and chemical environment, which can also give origin to unwanted parasite secondary reactions.

On the basis of these considerations, the inventors have identified the hypochlorites, preferably sodium, calcium and lithium hypochlorite, as a valid alternative to well-known oxidizers since, in the particular oxidation conditions that are commonly adopted in the process of post-use absorbent products, are respectful of the material treated and do not incur adverse secondary reactions.

The sodium, calcium or lithium hypochlorite (as well as for magnesium or other metals), once mixed with water dissolves dissociating itself in the ClO⁻ anion and respective metal cation (e.g. Na⁺) according to the reaction (1), taken as an example for sodium hypochlorite,

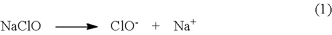

The hypochlorite anion ClO⁻ released and solvated by water molecules, as such, has a reduction potential associated with semi-reaction (2) below, equal to +0.90 V.

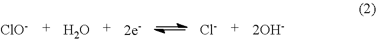 [SMILES: "ClO-" ; [Cl-] ; O]

At the same time, the hypochlorite anion ClO⁻ dissolved in water, partially hydrolyze, generating hypochlorous acid HOCl according to the acid-base balance reaction below (3).

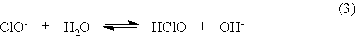

Hypochlorous acid HClO, unlike the respective conjugated CLO⁻ base, turns out to be a much stronger oxidizer, with a standard reduction potential of +1.63 V according to semi-reaction (4) described below

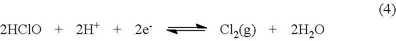

By virtue of its marked oxidizing nature, HOCl acts as a biocidal agent towards numerous pathogens (e.g. Escherichia coli) and as a decontaminating agent against possible organic residues, primarily of a metabolic nature, degradable by oxidative way. The sodium hypochlorite is in fact known in its uses as a whitening agent or for the water potabilization.

However, it is necessary to consider that hypochlorous acid is further involved in another redox balance (5) undesired and described below:

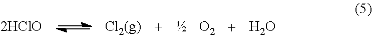

As can be seen from the reaction (5), hypochlorous acid as such goes to a dismutation reaction that involves its degradation to gaseous chlorine, oxygen and water. The greater the amount of HClO in aqueous solution and the more pronounced will be said chemical balance.

However, this reaction is strictly dependent on the pH. Acid environments, for instance, favoring the alkaline acid reaction (3), involve the increase in the concentration of HClO, the shift of the balance reaction (5) towards the products and consequently the unwanted increase in the decomposition of the hypochlorous acid.

Precisely for this reason, the commercial hypochlorite solutions are generically formulated and sold in alkaline conditions.

The addition of sodium hydroxide, i.e. NaOH, which is a strong base, inhibits the reaction (3) keeping low (but sufficient for oxidative purposes) the concentration of HClO and consequently limiting its degradation according to the reaction (5). The presence of the strong base therefore stabilizes the solutions of NaClO, conventionally of concentration not exceeding 15% m/v.

Sodium hydroxide is also used to slow down the decomposition of sodium hypochlorite in sodium chloride and sodium chlorate according to the dismutation reaction (6):

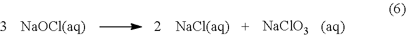

or to chloride based on the reaction (7):

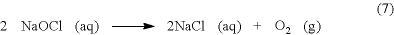

which proceeds on the basis of the temperature and/or pH conditions, and/or presence of metals as catalysts.

The use of hypochlorites as relatively mild oxidizers e.g. with a standard potential in aqueous solution of 1.7 V or lower, involves the great advantage of not de-polymerizing the SAP solubilizing it and, therefore, not affecting the yield and quality of recycled materials. The use of more aggressive oxidizers in aqueous solution excessively impact on the SAP which is therefore chemically decontaminated but on conditions that do not allow efficient and quantitative recovery.

### Grinding Step

The absorbent sanitary products, once used by consumers, are commonly thrown in a crumpled-up form for reasons of space, hygiene and possible bad smells. The compact form of the discarded sanitary article, however, tends to isolate the biological material inside, making it difficult to have it available for decontamination and sterilization actions.

Unlike WO2021130575A1, the present invention proposes to put a grinding of post-use absorbent sanitary products before any chemical or thermal treatment with the aim of increasing the surface of material exposed to decontaminating and sterilizing agents.

Therefore, the collection bags containing the post-use absorbent sanitary products are open and ground until reaching an average size lower than 40 cm, preferably lower than 30 cm, more preferably between 10 and 30 cm.

According to a preferred embodiment, the average size obtained is lower than or equal to 10 cm.

According to a further embodiment, it is possible to provide for at least two grinding steps for the progressive reduction of the average size obtained.

In addition, and preferably, the grinding(s) can all take place before oxidation, so that the grinder is not exposed to the chemical aggressiveness of oxidation.

Always in the perspective of economic convenience of the process, for grinding it is possible to use a common industrial grinder, for instance a grinder with a technology with knife rotor capable of guaranteeing average size of interest.

The grinding step prior to the decontamination treatment could give rise to odorous emissions due to the very nature of the products. Therefore, to avoid the spread of these emissions in the production environment and also outside, it is possible to provide that the grinding process takes place with the help of an aspiration system for the reduction of pollutants in the air, such as a scrubber, or a filter that uses a water flow in countercurrent referred to the direction of the current of polluted air which therefore undergoes a washing before being released into the atmosphere.

### Oxidation Step

The oxidation step, as described above, allows the decontamination of post-use absorbent sanitary products.

According to a preferred embodiment, said oxidation step can be carried out within a commercial autoclave.

Before the addition of oxidizer and always in a preferred embodiment, an initial depressurization between −0.9 bar and −0.7 bar can be applied which favors the subsequent absorption of the oxidative solution by the post-use absorbent sanitary materials. This lowering of the pressure can also afford the ground sanitary products within the autoclave to dry out, a condition favorable to the subsequent oxidative activity. In fact, the removal of water residues remained absorbed in the ground fragments of post-use absorbent sanitary products (even if partial) allows to avoid or in any case control the dilution of the oxidizer solution and therefore to guarantee better control on the concentration of added chemical agents.

The further advantage that follows the drying operation is a maximization of the absorption of the oxidizer aqueous solution by said sanitary products, improving in fact the efficiency of the oxidative treatment.

The oxidizer aqueous solution can be dosed by sprayers (pump system equipped with nozzles) which are able to uniformly impregnate post-use absorbent sanitary products according to a liquid/solid ratio included in an interval between 0.2-3, preferably between 0.8-2, preferably about 1 inside the autoclave.

The amount of oxidative aqueous solution is dosed in such a way that ground post-use absorbent sanitary products can incorporate/absorb/include this solution thus avoiding the formation of a suspension, i.e. a liquid/solid step separation, but effectively generating a moist solid impregnated with oxidizer.

Therefore, unlike the known art processes, in the present invention, post-use ground sanitary products are not immersed in any oxidizing bath but exclusively treated with a dosed oxidizing solution with the slightest amount of water necessary to dissolve the oxidizer and promote its oxidative action.

Preferably, the oxidizer solution is dosed at room temperature.

Preferably, the aqueous oxidizer solution is an aqueous sodium hypochlorite solution.

The aqueous sodium hypochlorite solution introduced can have a concentration by weight included in an interval between 6% and 15% m/v.

Preferably, the concentration by weight in the aqueous sodium hypochlorite solution is between 13% and 15% m/v.

The aqueous sodium hypochlorite solution can be dosed with a ratio of 0.01-0.5 moles of hypochlorite (meant as only ClO⁻ anion) per kilogram of post-use absorbent sanitary article,
preferably with a ratio of 0.02-0.3 moles of hypochlorite (ClO⁻) per kilogram of post-use absorbent sanitary article,
preferably with a ratio of 0.03-0.17 moles of hypochlorite (ClO⁻) per kilogram of post-use absorbent sanitary article.

The treatment of post-use ground absorbent sanitary products with the oxidizing solution lasts 3 hours, preferably 2 hours, preferably 1 hour, preferably less than one hour.

The treatment of post-use ground absorbent sanitary products with the oxidizing solution can be carried out by keeping the material in treatment in motion.

The treatment of post-use ground sanitary products with the oxidizing solution is performed at a temperature lower than 80° C., preferably lower than 70° C., preferably lower than 60° C., preferably lower than 50° C., preferably lower than 40° C., preferably lower than 30° C., preferably at room temperature.

According to further non-limiting embodiments, the oxidizer used is one among lithium hypochlorite, calcium hypochlorite, potassium hypochlorite, magnesium hypochlorite.

The aqueous hypochlorite solution used, preferably sodium hypochlorite, is strongly alkaline, with a pH≥13.

Since the pH of post-use absorbent sanitary products as such is around 7, it is clear that the addition of this oxidizing solution to the reaction medium makes the pH vary abruptly bringing it to values between 7.1 and 10, for instance between 7.1-7.5, 7.5-10, 8-10, 8.5-10.

Therefore, according to the present invention, the oxidation of post-use absorbent sanitary products is performed in alkaline conditions.

The alkaline conditions achieved are such as to guarantee adequate oxidizing activity by the hypochlorite without being involved in collateral reactions such as decomposition ones.

The sodium hypochlorite solution is sufficiently oxidizing to allow the decontamination from secondary metabolites excreted by the body but at the same time mild enough to prevent the materials of post-use absorbent sanitary products from being degraded, damaged or decomposed.

In particular, the hypochlorite solution, preferably sodium hypochlorite, allows to avoid the unwanted depolymerization of the super absorbent polymer (SAP).

In a form of non-limiting embodiment, the alkaline hypochlorite (NaClO, KClO, LiClO etc can be obtained in situ starting from the calcium hypochlorite (Ca(ClO)₂) by treatment with phosphorus salts.

Preferably, said phosphorus salts can be on orthophosphate (PO₄³⁻) or its conjugated acids such as hydrogen phosphate (HPO₄²⁻) or dihydrogen phosphate (H₂PO₄⁻).

Preferably, said phosphorus salts can be sodium or potassium salts.

According to a form of non-limiting embodiment, the oxidation step can be carried out within a special closed tank, under suction, located between the grinder placed upstream and an autoclave, used for the subsequent sterilization and drying steps, placed downstream.

### Inactivation Step

At the end of the steps previously described, post-use absorbent sanitary products now oxidized can however contain unreacted and locally concentrated residues. The latter can be responsible for a partial oxidation of the cellulose fiber to oxycellulose. In addition, a possible and excessive accumulation of alkaline substances due to the presence of bases used as stabilizers of some oxidizers, for instance hydroxides and carbonates for sodium hypochlorite and calcium hypochlorite, would make the waste of the process of obtaining secondary raw materials not compliant with Legislative Decree 62/2019 and therefore not acceptable in terms of End of Waste.

In fact, the secondary raw material subjected to release test must return an eluate that must have a 5.5<pH<9.5 (Table 4 of Legislative Decree 62/2019) To overcome this problem, it is possible to provide one or more inactivation steps downstream of the oxidation step, in order to avoid the oxidation of the cellulose to oxycellulose and allow the re-balancing for the pH in the secondary raw materials, as required by Legislative Decree 62/2019.

A first inactivation step relates to the inactivation of any oxidizer residues, preferably hypochlorite, still present at the end of the previous oxidation step. The residues of hypochlorite, if locally concentrated, could cause partial oxidation of the cellulose fiber to oxycellulose, involving a decrease in the mechanical resistance of the fiber itself and therefore influencing the quality and quantity of the recycled material.

The mechanical resistance of recycled cellulose fibers represents one of the crucial qualities sought by cellulose buyers. Reducing or deleting the presence of oxycellulose allows to considerably increase the value of the recycled material. This improvement contributes in a tangible way to the economic sustainability of the process, promoting a virtuous model of circular economy, wherein the materials are continually reused in high value applications.

Furthermore, this inactivation prevents the accumulation of unreacted oxidizer that would be potentially dangerous in the subsequent drying of the treated post-use absorbent sanitary products.

Said inactivation is performed by introducing a solution containing an antioxidant or reducing agent through sprayers placed inside the autoclave or, according to another embodiment, inside a special closed tank under suction wherein the oxidation step was performed.

The antioxidant solution can have a 5% concentration by weight compared to the weight in kg of used sanitary products (post-use) to be treated.

Preferably, the concentration of antioxidant is equal to 1% by weight compared to the weight in kg of the used sanitary products (post-use) to be treated.

As described for oxidative treatment, the quantity of added antioxidant solution is dosed in such a way as it can be absorbed by post-use ground sanitary products thus avoiding the formation of a suspension.

Antioxidants (or reducing agents) can be chosen by way of not limiting example between carboxylic organic acids and hydrogen peroxide.

Among the carboxylic organic acids, the chosen antioxidant can be by way of non-limiting example citric acid, malic acid, tartaric acid, ascorbic acid, oxalic acid or a combination thereof.

According to another embodiment, it is possible to use mineral acids in combination with non-acidic antioxidants. Mineral acids that can be used are, by way of non-limiting example: phosphoric acid, hydrochloric acid, sulfuric acid, or their properly dosed mixtures within the limits provided by the End of Waste standard (Legislative Decree 62/2019) for the release of chlorides and/or sulphates (respectively 1200 and 1000 mg/l).

The inactivation step with the aforementioned antioxidant agents is such as to lower the pH of the medium but always keeping it alkaline, i.e. greater than 7.

According to a preferred embodiment, the antioxidant chosen among organic acids is oxalic acid.

According to a particularly preferred embodiment, antioxidant is hydrogen peroxide (H₂O₂), which carries out its antioxidant/reducing action thanks to the presence of an alkaline pH.

If the oxidizer used in the oxidation step was stabilized with hydroxides and/or carbonates, particularly preferred as an antioxidant is hydrogen peroxide.

To the first inactivation step of the oxidizer, follows a second inactivation step of any alkaline residues deriving from the use of hydroxides and/or carbonates as stabilizers, e.g. for sodium hypochlorite. The agent for the neutralization of the alkaline pH can be added within the autoclave wherein the oxidation step was performed (or, according to another embodiment, within a special closed tank under suction) through sprayers that introduce an acidic solution with 5% by weight concentration compared to the weight in kg of used sanitary products (post-use) to be treated.

Preferably, the acidic concentration is equal to 1% by weight compared to the weight in kg of used sanitary products (post-use) to be treated.

According to a preferred embodiment, the inactivation of alkaline residues and oxidizer residues can be performed simultaneously using preferably an acidic antioxidant with 5% concentration by weight compared to the weight in kg of used sanitary products (post-use) to be treated wherein acidic antioxidant can be one between citric acid, malic acid, tartaric acid, ascorbic acid, oxalic acid, hydrogen peroxide.

Preferably, the concentration by weight of the acidic antioxidant compared to the weight in kg of the used sanitary products (post-use) to be treated is 1%.

Preferably, acidic antioxidant is oxalic acid or percarbonate, e.g. sodium percarbonate.

More preferably, acidic antioxidant is hydrogen peroxide.

The different inactivation steps depend on the type of hypochlorite used in the oxidation step. For instance, sodium hypochlorite, being very active, is marketed in concentrated solutions that do not exceed 15% m/v and are also stabilized with bases such as sodium hydroxide and/or sodium carbonate. Using sodium hypochlorite as an oxidizer, an inactivation step will be necessary both to eliminate any oxidizer residues and to eliminate the hydroxides and/or carbonates that would affect the quality of the raw material to be recycled.

If calcium hypochlorite is used as an oxidizer, which is less reactive than the previous one, the inactivation step will aim to eliminate mainly oxidizer residues. This is because the hypochlorite solution that would be prepared starting from the solid calcium hypochlorite sold as Ca(ClO)₂ at 65-70% would not contain the same quantities of alkaline compounds present in a solution of the same concentration of hypochlorite but prepared with sodium hypochlorite, instead sold as NaClO at 14-15%. On average, with the same moles of ClO⁻ used, the first will have a alkaline concentration (intended as total OH) at the maximum of a third compared to the second. Consequently, pH correction may not be necessary.

According to an embodiment, using sodium hypochlorite as an oxidizer, the subsequent inactivation can be carried out at different times first with an antioxidant and then with an acid.

According to a preferred embodiment, the inactivation step for the simultaneous neutralization of pH and alkaline residues can be performed using oxalic acid or hydrogen peroxide.

Using calcium hypochlorite, the subsequent inactivation step is performed only by neutralizing the oxidizer residue and inactivation with acid is superfluous.

In the case of the calcium hypochlorite, the concentration in aqueous solution can be between 15-30% m/v.

In a particularly preferred embodiment, the inactivating chemical agent for the simultaneous neutralization of the alkaline pH and oxidizer residues is hydrogen peroxide.

This agent for simultaneous inactivation combines a reducing activity, i.e. inactivation of the oxidizer residues, to an acid activity or lowering the pH of the material. The acidic character of the hydrogen peroxide solution remains only up to the mixing with the reaction medium. In fact, the acid-base reaction that is triggered between hydrogen peroxide and alkaline residues in the solution of the medium (e.g., hydroxides) is such as to lower the pH from a value next or greater than 10 to a preferred value around 8-9, thus reducing the alkalinity of the medium but still leaving the solution alkaline. It follows that the added hydrogen peroxide is no longer dissolved in an acidic medium but rather decidedly alkaline. In these alkaline conditions, hydrogen peroxide cannot act as an oxidizer and instead displays a reducing activity to neutralize the residual oxidizer, such as preferably hypochlorite, preferably sodium hypochlorite.

Said inactivation reaction of the unreacted hypochlorite by means of hydrogen peroxide generates chloride, with harmless chemical nature and elementary oxygen, which can be easily removed from the medium.

The inactivation agent, preferably in the form of aqueous liquid solution and preferably hydrogen peroxide, is dosed and added through the sprayers inside the autoclave or, according to another embodiment, within a special closed tank under suction wherein oxidation had previously occurred.

Preferably, the ratio between the moles of hydrogen peroxide and the moles of hypochlorite (to be intended as only the ClO⁻) is between 0.05-2.00, preferably 0.1-1.3, preferably 0.4-0.8.

Preferably, the inactivating solution is added at room temperature.

Preferably, the inactivation reaction of the residual alkalinity and unreacted oxidizer is performed at room temperature.

Preferably, the inactivation reaction of the residual alkalinity and unreacted oxidizer is performed in motion.

Preferably, the inactivation reaction of the residual alkalinity and unreacted oxidizer is performed at ambient pressure.

Preferably, the inactivation reaction of the residual alkalinity and unreacted oxidizer leads the pH to a value between 8-9.

Preferably, the added solution is left to act for 1 hour, preferably for 45 minutes, preferably for 30 minutes, preferably for 20 minutes, preferably for 15 minutes, preferably for 10 minutes, preferably for 5 minutes, preferably for 1 minute.

According to an embodiment, the inactivating agent is sodium percarbonate.

### Sterilization Step

According to a preferred embodiment, ground and decontaminated post-use absorbent sanitary products are sanitized/sterilized for the removal of pathogens.

In the procedures described by known art, post-use absorbent sanitary products are commonly sterilized in a crumpled-up and closed form. In not preliminarily ground products, the waterproof plastic film present on the external surface of these sanitary products acts as a sealing and insulating agent, significantly complicating sterilization operations. In fact, if not appropriately torn, this film acts as a thermal insulator, opposing to heat and thus making more necessary thermal sterilization cycles (at least 3). Moreover, the presence of the external plastic film, if intact, involves the use of high temperatures and long sterilization times in order to have the certainty that the thermal energy can penetrate deeply in the crumpled up post-use absorbent sanitary products. Hence therefore follows greater energy consumption.

Instead, according to the present invention, the sterilization of post-use absorbent sanitary products provides that the grinding step is carried out upstream, thus allowing to reduce temperature and sterilization times.

The sterilization is performed inside a heated autoclave, where said sterilization takes place at a temperature lower than 134° C., preferably lower than 125° C., preferably 120° C. and at a pressure lower than 3 bars, preferably lower than 2 bars, preferably 1 bar for a period of time of at least 5 minutes, preferably at least 7 minutes, preferably at least 10 minutes, preferably at least 12 minutes, preferably at least 15 minutes, preferably exceeding 15 minutes.

This sterilization takes place by dispensing saturated steam in direct contact with the ground post-use absorbent sanitary products and preferably already treated with oxidizer and neutralizer.

Preferably, this sterilization is performed in motion.

Preferably, a single thermal cycle is sufficient for the sterilization of said sanitary products.

Preferably, before loading post-use absorbent sanitary products in autoclave, the latter is depressurized for air removal.

According to a non-limiting embodiment, sterilization is performed prior to the oxidation and neutralization steps but always after the grinding of post-use absorbent sanitary products.

According to this embodiment, at the end of the sterilization step, the temperature reached by the ground and sterilized post-use absorbent sanitary products would be such as to cause the decomposition of the oxidizer in the subsequent oxidation step, with the consequent oxidation of the contained cellulose fiber in these absorbent sanitary products. This phenomenon can occur for temperatures above 90° C.

To avoid the oxidation of cellulose fiber, which would affect the quality of the recycled material, it is possible to provide a cooling step down to a temperature of at least 80° C. before subjecting said material to chemical oxidation.

According to a preferred embodiment, the cooling step involves bringing the temperature of the sterilized material to 70° C.

According to a further preferred embodiment, the cooling step involves bringing the temperature of the sterilized material to 60° C.

The execution sequence of oxidation, inactivation and sterilization steps is not binding.

In a preferred embodiment of the present invention, oxidation, inactivation and sterilization take place in sequence within the same autoclave.

In another embodiment, sterilization can be performed prior to oxidation and inactivation.

In a further embodiment, sterilization can be performed between oxidation and inactivation.

In a still further embodiment, sterilization can be performed simultaneously with oxidation or inactivation.

According to a non-limiting embodiment, the autoclave includes a non-sticking coating, such as for instance silicone, Teflon and other materials with non-sticking properties, capable of offering autoclave protection against corrosion phenomena capable of preventing cellulose and super-absorbent material from remaining attached to the internal surface of the autoclave during the subsequent drying step.

### Drying Step

Once the sterilization is complete, a partial drying step of post-use absorbent sanitary products can be provided to restore the residual humidity to the original level of the decontaminated product before sterilization itself.

Preferably, the residual humidity level is lower than 80% m/m (intended as a percentage ratio of the mass of water, expressed in grams [g], contained in 100 grams of treated material), preferably lower than 70%, preferably lower 65%, preferably 60%.

Said partial drying can be performed inside the autoclave via heated shell and application of the void for a time of less than 60 minutes, preferably less than 45 minutes, preferably less than 30 minutes, preferably less than 20 minutes, preferably 15 minutes, preferably less than 15 min, preferably less than 12 minutes.

Then, post-use absorbent sanitary products are subjected to a real drying step that can be performed in a dryer by introducing hot air for a time of at least 2 minutes, preferably at least 5 minutes, preferably of at least 10 minutes, preferably at least 15 minutes by which the residual humidity is brought to a value of less than 50% by weight, preferably less than 40%, preferably less than 30%, preferably less than 20%, preferably less than 10%, preferably less than 7%, preferably 5%.

### Separation Step

Once dried, the post-use absorbent sanitary material is sent to at least one separator, e.g. a centrifuge, for the separation of plastic from a composite material containing or consisting of cellulose and/or SAP. The centrifuge, taking advantage of a different density of the materials, therefore allows the separation of the plastic component from the non-plastic component, typically, from the SAP+cellulose aggregate.

The SAP+cellulose aggregate is in fact a cellulose product with a variable percentage of SAP, which will be function of the initial quantity of SAP contained in the post-use absorbent sanitary articles and from the separations made downstream of the process. This composite product can be recovered in the forms of:
Cellulose with high SAP content
Cellulose with low SAP content
   Cellulose with High SAP Content

The high SAP cellulose obtained by separation of the plastic material can contain a percentage of super absorbent polymer (SAP) of the order of 30-40%, for instance lower than or equal to 40%, preferably lower than 35%, preferably around 34%.

Preferably, said high-SAP cellulose can contain a percentage of residual humidity of less than 20% m/m.

Preferably, said high-SAP cellulose can contain a percentage of foreign materials (other than SAP and cellulose) lower than or equal to 5% m/m (evaluated on dry material, i.e. without water).

Preferably, the ability to absorb water of said high-SAP cellulose is greater than 10 times its mass.

Said high-SAP cellulose, as regenerated material, can be used as such for the preparation of new absorbent sanitary products or other similar products.

Cellulose with Low SAP Content

The SAP+cellulose aggregate with a high SAP content can undergo a further separation process (e.g. through rotating mechanical separators with drilled fixed filter sects) for the partial or total removal of the super absorbent polymer content, thus generating cellulose with a low SAP content.

In fact, by exploiting the different density between the cellulose (with a cotton appearance and consistency) and the SAP (with a grainy, sandy consistency) it is possible to induce the detachment of the two components.

The low-content cellulose obtained by partial separation of the SAP from cellulose can contain a percentage of super absorbent polymer (SAP) lower than or equal to 5%, preferably at 3%, preferably at 1%.

Preferably, said low SAP content cellulose can contain a percentage of residual humidity lower than 20% m/m.

Preferably, said low SAP content cellulose can contain a percentage of foreign materials (other than SAP and cellulose) lower than or equal to 5% m/m (evaluated on dry material, i.e. without water).

Preferably, the ability to absorb water of said low SAP content cellulose is greater than 5 times its mass.

Said cellulose with a low SAP content, as regenerated material, can be used as such for the preparation of new absorbent sanitary products or other more or less similar products, or as a building block for biochemistry.

According to a further embodiment, said process of further separation allows to obtain substantially pure cellulose, that is, substantially without SAP and the SAP recovered can be recovered almost quantitatively with respect to the absorbent sanitary article before its use.

### Recovered SAP

This process of further separation allows in parallel to obtain pure super absorbent polymer (SAP), whose recovery will depend on the number of separation cycles made on the SAP+cellulose composite material.

Advantageously, the super absorbent polymer is recovered exclusively mechanically at the end of a thermo-chemical process totally respectful of its structural characteristics, without being altered or modified its technical properties. The recovered SAP, having therefore maintained its substantially unchanged chemical-physical characteristics, can be considered as the original material. In fact, unlike what is reported in the well-known art, the super absorbent polymer is never subjected to excessively oxidizing environments such as to induce its depolymerization in oligomeric fragments (e.g. linear polyacrylate) soluble in aqueous environments.

Since in the process according to the present invention, no solubilization of the SAP, or its unwanted depolymerization, occurs and said super absorbent material can be totally recovered either in pure or aggregated form to cellulose in the SAP+Cellulose composite material in variable percentages.

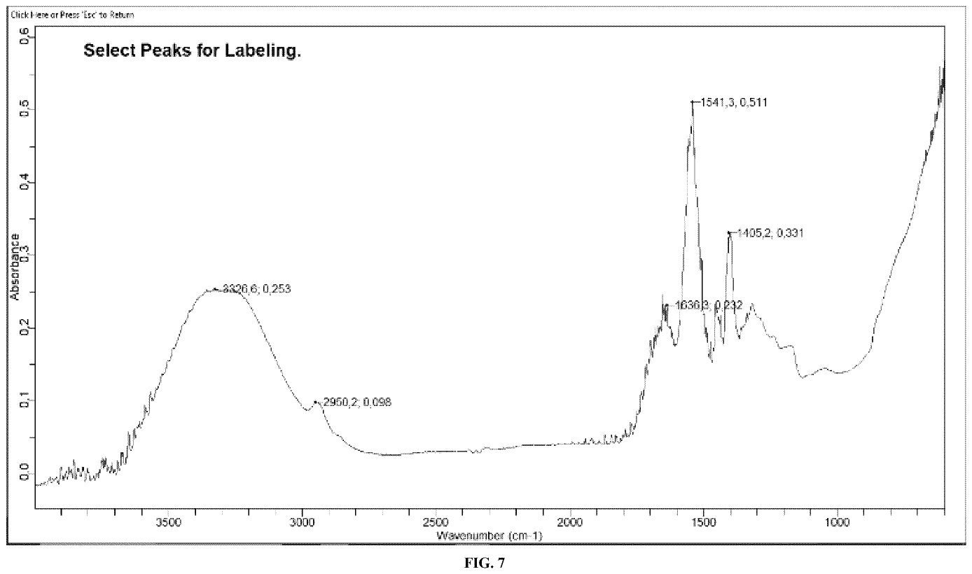

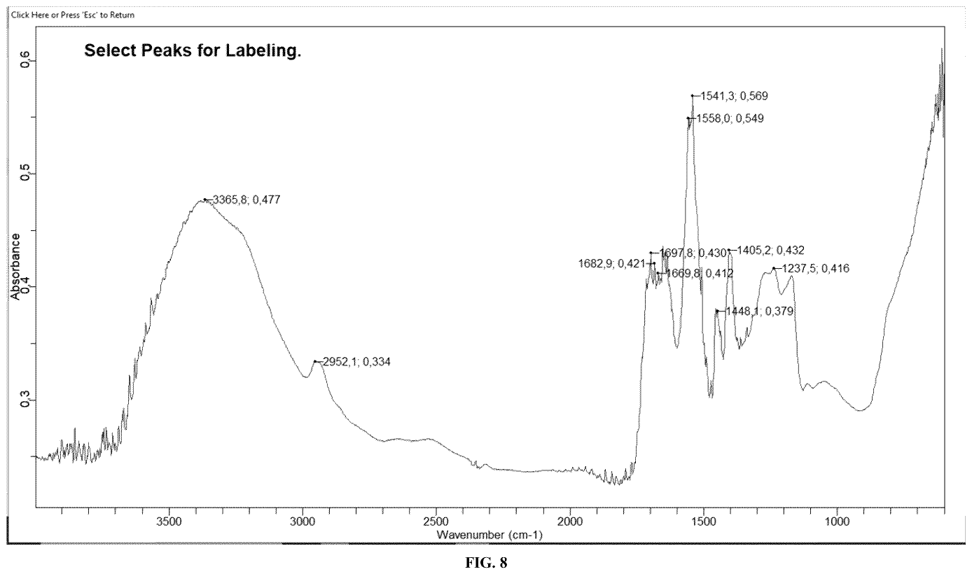

To confirm the integrity and quality of the SAP recovered after the oxidation-sterilization process, a FTIR in ATR (Attenuated Total Reflectance) spectrum was recorded (FIG. 8) to compare it with a reference spectrum of pure SAP. (FIG. 7) The two spectra are consistent and superimposable (the broad band over 3000 cm⁻¹, as evident to the expert in the field, is caused by the stretching of water molecules absorbed by humidity), once again remarking as the entire thermo-chemical process according to the present invention is respectful of the chemical-physical properties of the treated materials.

The cellulose, the SAP and the plastic component that make up the absorbent products are recovered in a quantitative way, preserving their structural integrity and the main chemical-physical characteristics.

### Recovered Pure Cellulose

For the recovered cellulose fiber, there is a decrease in apparent density (as defined in the paragraph “determination of the apparent density of cellulose fiber” in the “analytical methods” section) and an increase in the specific surface. The pure one used to pack absorbent sanitary products has a specific surface of 46 m²/g, consistent with that of the common cotton fibers [1], and an apparent density of 720 mg/ml. Pure cellulose fiber (% SAP and % plastics <1%, by weight) recovered post-treatment with hypochlorite according to the present invention, on the other hand, has an apparent density, for instance, not exceeding approx. 518 mg/ml (contamination higher than or equal to 1% by weight of SAP would lower the apparent density value), and a specific surface for instance of approx. 107 m²/g.

The type of cellulosic material obtained post-treatment with the aforementioned chemical-physical characteristics makes itself good for some of the purposes indicated by Legislative Decree 62/2019, art. 4, paragraph 3, as construction materials, such as thermal and acoustic insulating. Similarly to the cotton textile waste (CTW, Cotton Textile Waste), the cellulose recovered according to the present invention can be used as a base to obtain other composite materials based on low density cellulose and greater specific surface, which, in addition to being totally biodegradable, are low cost.

An advantageous aspect is to be able to obtain again the main raw materials used for the realization of the sanitary products quantitatively and substantially separated or separable.

Therefore, the method of the invention allows to obtain, separated or separable from each other: plastics and at least one between SAP and cellulose, wherein plastics and at least one between SAP and cellulose have substantially the same characteristics as the starting products, before being subjected to the treatments of the invention.

It has been experimentally verified that after the treatments of the invention cellulose does not degrade and maintains its apparent density which preferably is only lower than 50%, preferably 45%, preferably 40%, preferably 35%, preferably 30%, preferably 25%, preferably 20% of the apparent density of the cellulose present in sanitary products before the treatment according to the present invention.

It was also experimentally verified that after the treatments of the invention cellulose does not degrade and appears to have a specific surface higher than that of the cellulose present in the sanitary products before the treatment according to the present invention.

The increase in the specific surface is equal to at least 10%, 20%, 30%, 40%, 50%, 60%, 70%, 80%, 90%, 95%, 100%, 110%, 120%, 130%, 140%, 150%, 160%, 170%, 180%, 190%, 200% or higher than 200% compared to that of the cellulose present in sanitary products before the treatment according to the present invention.

Equally advantageously it was found that SAP remains unchanged after the treatments and is recoverable almost 100% by mechanical separation from cellulose.

In the following, not limiting embodiments of the invention are described, making reference to the attached drawings.

### Method 1

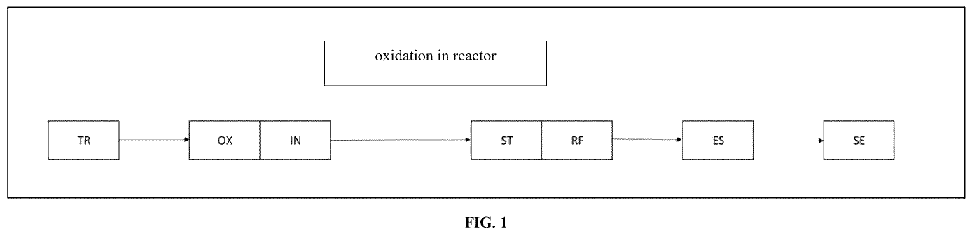

On the basis of the steps described above, it is possible to create a preferred method for the recovery of materials constituting the absorbent sanitary products contaminated according to the exemplary scheme represented in FIG. 1.

A first step of the method provides for the grinding TR of the post-use absorbent sanitary products obtained within a grinder until the desired average size is achieved in order to facilitate subsequent stages by reducing its time and consequently energy consumption.

It is followed by an oxidation step OX intended to decontaminate the ground absorbent sanitary products by introducing the oxidizer in aqueous solution in the reactor through the aid of sprayers.

According to another form of non-limiting embodiment, the oxidation step can be carried out within a special closed tank, under suction, located between the grinder placed upstream and an autoclave, used for the subsequent sterilization and drying steps, placed downstream.

Subsequently there is an inactivation step for the elimination of the local accumulations of unreacted oxidizer and the neutralization of alkaline residues. The two operations can be carried out at the same time or separately depending on whether the reducing agent has or does not also have acidic properties.

A step ST follows to sterilize the ground and chemically treated post-use absorbent sanitary products for the reduction of present pathogenic microorganisms.

A cooling step RF instead aims to lower the temperature of post-use absorbent sanitary products after the sterilization step ST, bringing it back to the ambient one.

Subsequently, as reported in FIG. 1, a step ES is provided to dry thermo-chemically treated products inside a dryer with a temperature between 110° C. and 130° C. for a period of about 2 hours which will allow to leave the color of the material unchanged, e.g. white, as well as in entry to this method.

Finally, as a last step, there is a separation step SE of the plastic component from the SAP+cellulose composite product, performed through separators such as rotating centrifuges, with a fixed drilled screen.

The method described allows to substantially recover 100% of the super-absorbent polymers (SAP), or in pure form or combined with cellulose, since hypochlorite does not depolymerize SAP solubilizing it in water, as happens due to the action of other oxidizers such as hydrogen peroxide (used in acid conditions as oxidizer) and/or potassium peroxymonosulfate and/or potassium and/or sodium persulfate.

### Method 2

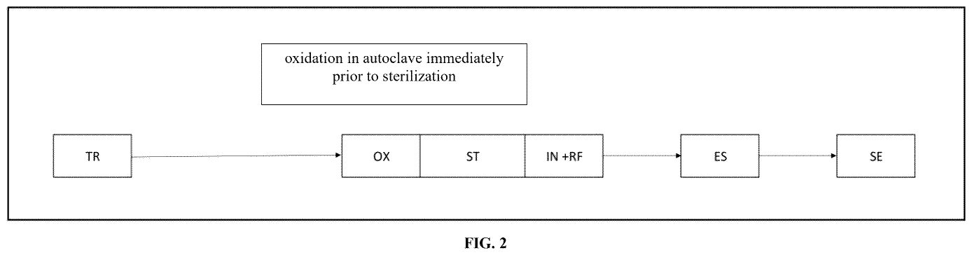

In FIG. 2 the exemplary scheme of a further embodiment for the recovery of materials constituting the contaminated absorbent sanitary products is shown.

A first step TR of the method provides for the grinding of the post-use absorbent sanitary products put within a grinder until the desired average size is achieved in order to facilitate subsequent stages by reducing its times and consequently energy consumption.

Subsequently, the oxidation OX and sterilization ST steps are made in close sequence (OX+ST) in the autoclave for an oxidative decontamination of ground absorbent sanitary products and a reduction of pathogenic microorganisms.

This combined treatment is preferentially carried out at temperatures lower or equal to 90° C., in order to avoid compromising the quality of the recycled material due to the phenomenon of oxidation of cellulose fiber contained in absorbent sanitary products.

Subsequently, this process method provides that the inactivation step IN for the neutralization of base and oxidizer residues takes place simultaneously with the RF cooling step (IN+RF). The neutralization operation, as for the realization according to method 1, may provide for a simultaneous acidic and reducing action by the selected chemical agent or a distinct treatment made by individual agents added in sequence or at the same time.

Subsequently, as reported in FIG. 2, a drying step ES is expected of thermo-chemically treated products inside a dryer with a temperature between 110° C. and 130° C. for a period of about 2 hours which will allow to leave the color of the material unchanged, e.g. white, as well as in entry to this method.

Finally, as a last step, there is a separation step SE of the plastic component from the SAP+cellulose composite product, made by separators such as rotating centrifuges, with a fixed drilled screen.

### Method 3

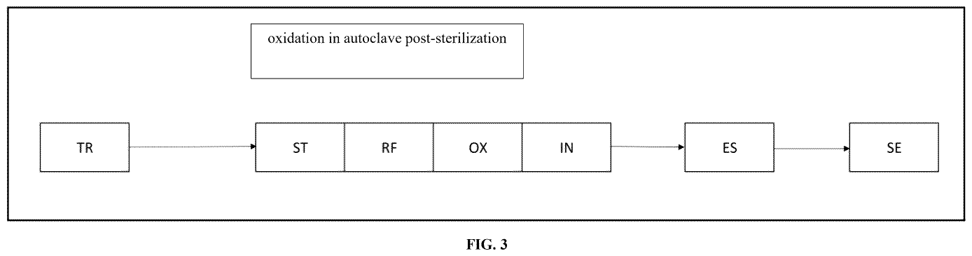

A further embodiment of the present invention follows the process scheme shown in FIG. 3.

A first step of the method provides for the grinding step TR of the post-use absorbent sanitary products put inside a grinder until the desired average size is achieved in order to facilitate subsequent steps by reducing its times and consequently energy consumption.

Then there is a step ST to sterilize the post-use ground absorbent sanitary products in order to eliminate present pathogenic microorganisms.

According to a preferred embodiment, the sterilization step also includes a step of introducing steam on sterilized and ground absorbent sanitary products.

The subsequent cooling step RF then aims to lower the temperature of post-use absorbent sanitary products after the sterilization step ST down to a temperature to which the decomposition phenomenon of an oxidizer cannot occur, when introduced inside the autoclave, whose reaction at temperatures above 90° C. would compromise the quality of the recyclable material due to the oxidation phenomenon of cellulose fiber contained in absorbent sanitary products.

The subsequent oxidation step OX is intended to decontaminate the ground and sterilized absorbent sanitary products by introducing the oxidizer in aqueous solution in the autoclave through sprayers.

In the subsequent inactivation step IN, one or more inactivation steps are performed for the elimination of oxidizer residues and for the rebalancing of pH.

A drying step inside a dryer with a temperature between 110° C. and 130° C. for a period of time of about 2 hours will allow to leave the color of the material unchanged, e.g. white, as well as in entry to this method.

### EXAMPLES

### Analytical Methods

All post-use materials were found at the waste recycling plant of Contarina SpA-Treviso. All pure materials have been recovered for mechanical separation of commercially available diapers, adult diapers and/or traverses for incontinence, under the Pampers®, Chicco®, Huggies® or Tena® brand, with the help at the most of a vibro-sifter for the separation of granular SAP.

### Determination of Humidity.

The samples were dried in a stove up to constant weight, at 105±2° C. according to the UNI EN 15414-3: 2011 method—Determination of the humidity content by means of drying in a stove, or by the UNI 10667 method-16-2015 A—A gravimetric method for determining the residual humidity in post-use materials. The percentage value of the water content of any of the weight materials P° is obtained from the ratio between (P°−P)/P°, % (weight/weight), with the P value of the weight of the dried material up to constant weight. As a rule, the pre-weighted sample in a container with known tare is kept in a stove at 105° C. for two hours. After 2 hours, the P₁ weight value is determined. The sample is put in a stove for another 2 hours, and after 2 hours the value of the P₂ weight at room temperature is determined. If the difference between P₁ and P₂ is greater than the sensitivity of the balance used, the sample is put again in a stove. The weight of the sample placed n times in a stove, until the difference in weight between Pₙ₋₁ and Pₙ will be equal to the balance sensitivity, at room temperature. In the discussed examples sensitivity is ±0.01 g.

### Determination of SAP Percentage.

A well-known mass of a dry sample containing SAP is treated at 85-95° C. in a 10% (m/v) hydrogen peroxide solution, with a liquid/solid ratio 25 ml/g, for 20-30 minutes, and up to the dissolution of SAP, as reported by the method indicated in WO 2021/130575 A1—A method for separating and recovery super-absorbent polymers (SAP) from post-use absorbent sanitary products. The percentage value of the SAP content of any of the weight materials P° is obtained from the ratio (P°−P)/P°, % (weight/weight), with the value of the P weight of the recovered post-treatment material with hydrogen peroxide, filtered, washed with hot water and dried up to constant weight.

### Determination of Polyolefins Percentage.

A well-known mass of a dried sample containing “foils” of polyolefins is treated under reflux in xylene in a Soxhlet extractor, with a liquid/solid ratio equal to at least 10 ml/g, for 24 hours, as reported by the UNII ISO 6427: 2013 method, Plastics: Determination of Matter Extractable by Organic Solvents. The percentage value of the polyolefins content of any of the weight materials P° is obtained from the ratio (P°−P)/P°, % (weight/weight), with the weight value P of the recovered post-treatment material with xylene and dried up to constant weight.

### Determination of the Ability to Absorb Water.

A well-known mass of a dried sample containing SAP is suspended in an excess of water, with a liquid/solid ratio of at least 1000 ml per gram of material with a high SAP content. There must be a clear separation step. After 10 minutes everything is filtered in a pre-weighted basket, leaving to drip for 15 minutes, as shown in the NF V19-002: 1993-02-01. Determination of absorbent power, water retention capacity.

To evaluate the amount of water absorbed by the absorbent material compared to the initial weight P°, the ratio (P−P°)/P°, % (weight/weight) is calculated, with P weight value of the post-filtration recovered material subtracting the weight of the basket.

### Determination of the Apparent Density of Cellulose.

The apparent density can be calculated with the volumetric method. A well-known mass W of cellulose (dry or with a predetermined percentage of humidity) is placed in a graduated container of volume V, put on a balance. The balance is calibrated to zero, and water is added up to the score of volume V. The weight of the added water and the temperature are recorded.

The density value D is calculated as:

$$D = W / {V}_{A} , g / ml$$

Wherein W is the weight of the cellulose expressed in grams, and VA is the apparent volume of cellulose, in ml, evaluated by the relation:

$${V}_{A} = V - {W}_{w} / {D}_{w} , ml$$

Wherein Ww is the weight of the water added until the correspondence of the score of the container to volume V and D_{W} is the density of the water at room temperature (1.00 g/ml at 25° C.).

It is underlined, for instance, that for the evaluation of the apparent volume of cellulose fiber, a 500 ml container was necessary for 10 g of said material.

### Determination of the Specific Surface of Cellulose Fiber.

The specific surface is defined as the accessible surface of a solid per unit of mass. For cellulose fibers it can be determined with the colorimetric method of Methylene blue, a dye that has a molecular absorption spectrum with a maximum centered at 664 nm.

In order to determine this parameter, known quantities of cellulose fiber are placed in equilibrium for 24 hours with 5 dye solutions with different concentration, C1, C2, C3, C4 and C5. After 24 hours, the solid is separated from the solution by filtration, and the volume and concentration C of the non-absorbed residual dye is determined. The concentration, known the molar extinction coefficient of Methylene blue ε, is obtained through spectrophotometry in the visible, measuring absorbance A at 664 nm, A⁶⁶⁴. C is obtained from the relation A⁶⁶⁴=ε×b×c (b=1 cm). The value of F is obtained using the same relation but preparing solutions with known dye concentrations, in the interval from 1 to 30 mM.

To calculate the specific surface of cellulose, S, the Langmuir linearized equation is used:

$$C / {N}_{i} = C / {N}_{m} + 1 / ( {KN}_{m} ) \qquad ( 8 )$$

wherein C is the concentration of dye at the equilibrium achieved in correspondence with the dye solutions with different concentration Cᵢ (with i from 1 to 7) and Ni is the number of moles of Methylene blue absorbed per gram of cellulose fiber, calculated, for each concentration Cᵢ, as:

$${N}_{i} = ( V ( {C}_{i -} C ) ) / W with i from 1 to 7 \qquad ( 9 )$$

wherein Cᵢ is the initial analytical concentration and C that of the corresponding solution wherein a known mass W of fiber is immersed for 24 hours.

The Langmuir linearized equation (8) allows to obtain the 1/KNₘ intercept and the slope 1/Nm, with K constant and Nm the number of moles of Methylene blue absorbed per gram of cellulose fiber necessary to form the monolayer. The value of S will be given in m²/g from the Gregg equation [10]:

$$S {m}^{2} / g = {N}_{m} \times {N}_{A} \times α \times 1 {0}^{- 20} \qquad ( 10 )$$

wherein NA is Avogadro number, equal to 6.022×10²³ mol⁻¹, α the surface area occupied by Methylene blue, equal to 197.2 Å² and Nm the aforementioned number of moles of Methylene blue absorbed per gram of cellulose fiber.

The examples reported below are to be considered merely exemplary and not limiting the scope of the present invention.

Tests on post-use cellulose combinations (CE PC) were carried out, mixed with post-use super-absorbent polymer (SAP PC), and post-use polyolefins (PO PC), where said combination simulates the material present in the contaminated absorbent products that are intended to be recycled, for the evaluation of the efficiency of chemical and recycling treatment.

### Example 1

A laboratory sample was prepared as follows: 100 g (22 g of CE PC+7 g of SAP PC+1 g of PO PC+70 g of water), materials already subjected to sterilization in autoclave.

The sample was then treated with 90 ml of ACE® (Procter & Gamble) with 3% active chlorine, without carrying out the neutralization with the addition of one or more acids. This results in a sodium hypochlorite/solid ratio equal to 9.0%, w/w, and a liquid/solid ratio equal to 3 (ml/g), wherein the liquid, is the aqueous hypochlorite solution.

The test carried out at pH=13 performed with ACE® gave the expected effect of decontamination+bleaching, with 10% active chlorine concentration compared to dry (but with a liquid/solid ratio of 3 ml/g), and with a 10-15 minutes contact time.

The neutralization of the cellulose pulp was subsequently made with 10% by weight hydrogen peroxide, to inactivate the residual sodium hypochlorite. As a result, the material remains white and odorless (but the L/S ratio rises to 3.5), and with a contact time of at least 15 minutes.

The subsequent treatment in forced convection stove at 110° C. returns the white material like the one at the entrance. The pulp and waste recovered comply for pH, as well as for chlorides and sulphates (respectively lower than 1200 and 1000 mg/l).

The stove drying leaves the color unchanged, even if it dries at 110° C. for 15 hours. The browning is practically absent.

The above decontamination effect was evaluated on the dried and bleached sample. Quantities of dry sample have been submitted to release tests in appropriate solvents according to the official methods, and the extracts have been analyzed in order to verify the concentrations of the indicators (the chemicals that the legislator, or a method, or an entity designate as indicators of the degree of contamination of a system) referred to Legislative Decree 62/2019, Table 3B and Table 4. All the chemical criteria referred to in Table 3b and 4 are satisfied.

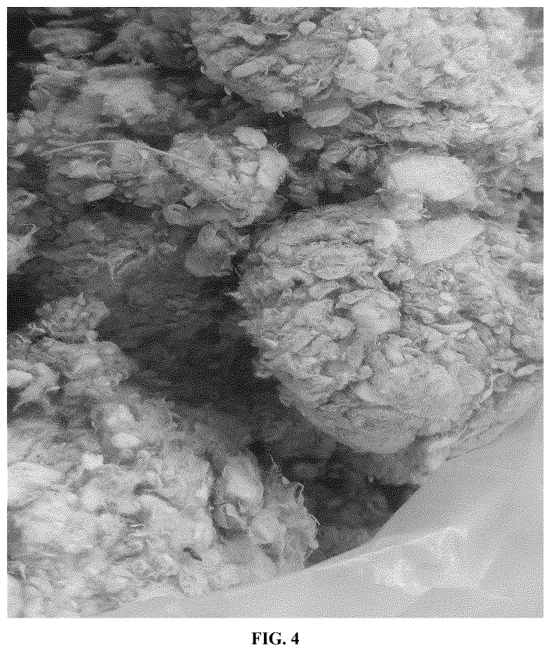

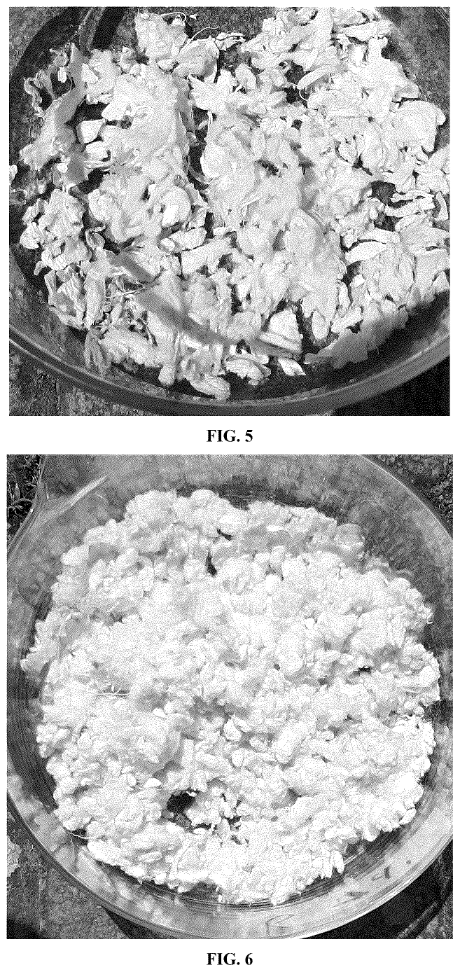

The whitening evaluation is visual. The contaminated material has different mixed shades, as can be seen from the different shades of grays in FIG. 4. After treatment the cellulosic material is white, apart from some dark fragments due to the residual plastic component (FIG. 5 and FIG. 6).

### Example 2

A laboratory sample was prepared as follows: 100 g (22 g of CE PC+7 g of SAP PC+1 g of PO PC+70 g of water), materials already subjected to sterilization in the autoclave. The sample was then treated with 90 ml of 3% active chlorine ACE®, without carrying out the neutralization with the addition of one or more acids. This results in a sodium hypochlorite/solid solution ratio equal to 9.0%, w/w, and a liquid/solid ratio equal to 3. The test carried out at pH=13 performed with ACE® has given the expected effect of decontamination+whitening with an active chlorine concentration of 10% compared to the solid, i.e. weight in kg of used sanitary products (post-use) to be treated (but with a liquid/solid ratio equal to 3), and with a contact time of at least 10-15 minutes. The neutralization of the cellulose pulp was subsequently made with 2.5% by weight oxalic acid, to inactivate the sodium hypochlorite residual, keeping alkaline the pH of the medium. The material remains white and odorless (but the L/S ratio rises to 3.5 for the addition of the acid solution), and with a contact time of at least 15 minutes. The subsequent drying treatment returns the white material like the one at the entrance in the stove. The pulp and waste recovered comply for pH, as well as for chlorides and sulphates (i.e. lower than 1200 and 1000 mg/l) respectively. Forced convection stove drying leaves the color unchanged, even if it dries at 110° C. for 15 hours. The browning remains even less mildly located on the external surface of the cellulose fragments. The “heart” of the flakes remains white, and the same retains its softness.

### Example 3

A laboratory sample was prepared as follows: 100 g (22 g of CE PC+7 g of SAP PC+1 g of PO PC+70 g of water) already sterilized. It was treated with 90 ml of 3% active chlorine ACE®, without carrying out neutralization with the addition of one or more acids. This results in a sodium hypochlorite/solid solution ratio equal to 9.0%, w/w, and a liquid/solid ratio equal to 3. The test carried out at pH=13 performed with ACE® gave the expected effect of decontamination+bleaching with an active chlorine concentration of 10% compared to dry (but with a liquid/solid ratio of 3), and with a contact time of at least 10-15 minutes. The neutralization of the cellulose pulp was subsequently made with 10% by weight citric acid, to inactivate the residual sodium hypochlorite while maintaining alkaline the pH of the medium. The material remains white and odorless (but the L/S ratio rises to 3.5 for the addition of the acid solution), and with a contact time of at least 15 minutes. The subsequent drying treatment at 110° C. returns the almost white material as the one at the entrance. The pulp and waste recovered comply for pH, as well as for chlorides and sulphates (i.e. lower than 1200 and 1000 mg/l) respectively. A stove drying leaves the color unchanged, even if it dries at 110° C. The “heart” of the flakes remains white, and the same retains its softness. Similar results were obtained with tartaric acid.

The previous examples use sodium hypochlorite, usually available in aqueous solution. The same results are however obtainable with other hypochlorites since the cation does not take part in any oxidation reaction.

In the previous examples, hypochlorite is provided in aqueous solution and the moles of hypochlorite in this solution are considered for the relationship with the kg of products to be treated and the volume of this solution is considered for the 1/kg relationship. When hypochlorite is provided directly in aqueous solution, it is possible that the latter present the quantity and concentration in solution suitable to meet both the mol/kg and 1/kg requirements for a given weight of used products (post-use) to deal with. If, on the other hand, the aqueous solution meets the mol/kg requirement but is too concentrated, water is added to also comply with the 1/kg requirement.

If the calcium hypochlorite powder is used to treat a given weight of used sanitary products (post-use), through appropriate conversions a quantity of powder is weighed according to the mol/kg requirement and a quantity of water is added on the basis of the 1/kg requirement.

### Example 4

Verification of the stability of SAP to oxidative treatment in the conditions of the invention. Study case: oxidation with NaClO at alkaline pH, followed by reduction with hydrogen peroxide in an alkaline environment.

A ground laboratory sample was prepared as follows: 20.2 g of pure cellulose+9.1 g of pure SAP+1.1 g of polyolefins+70.0 g of water. The sample constituted by mixing the three components was sprinkled with water to mix it and to make it similar to the real material, with average humidity 65-70%. All the water is absorbed by the material. The 69.7% wet material was then treated first with NaClO in alkaline pH conditions in a reactor with a hypochlorite/kg of sample molar ratio equal to 0.16 mol/kg, and then with hydrogen peroxide, always in alkaline pH conditions and with a molar ratio equal to 0.4% (peroxide vs hypochlorite). The two solutions were sprayed on the material. The material was then placed in the autoclave and three cycles at 2.1 bar and 134° C. were performed. The sample extracted from the autoclave (ICAN-Clave® model) was dried and subjected to mechanical separation by different metal mesh sieves covering the range included from 2 mm to 150 μm. The stacked sieves were placed on a vibro sifter (Retsch®), which made it possible to split the SAP grains, of dimensions smaller than a millimeter, from cellulose flakes and plastic strings, larger than a millimeter. A minimum contamination of the SAP by small cellulose fibers is not excluded. A minimum contamination of the cellulose is not excluded by small grains of SAP remained trapped in the flakes. The three fractions in the extent of 20.3 g of cellulose probably partially contaminated by SAP, 8.5 g of SAP and 1.0 g of polyolefins, with a mass loss of 2%, in line with the process losses that are estimated as negligible.

The identity of the three isolated fractions was confirmed by analysis performed by ATR spectrophotometry.

FIG. 8 shows the spectrum relating to the fraction of SAP recovered. The spectrum is consistent and superimposable with that of the pure SAP of FIG. 7.

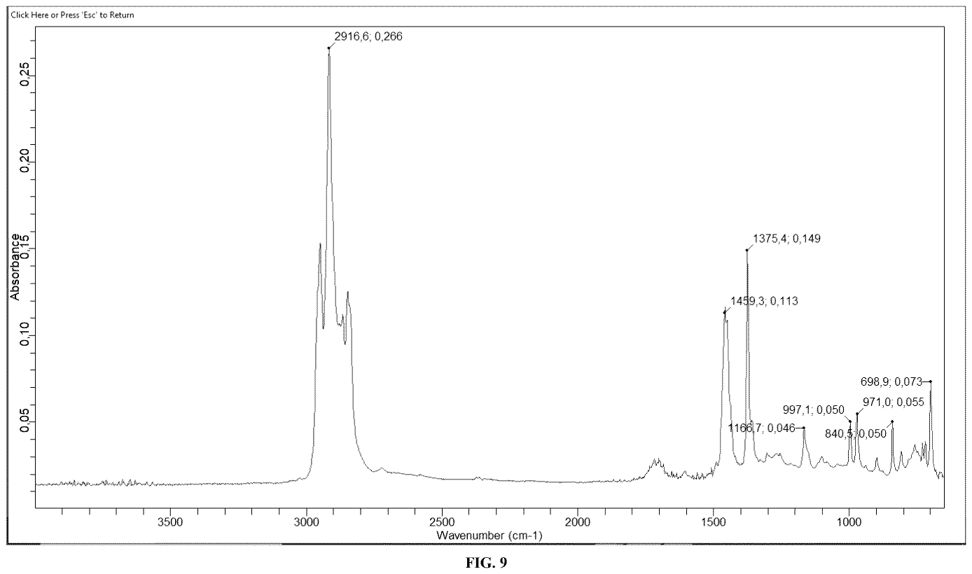

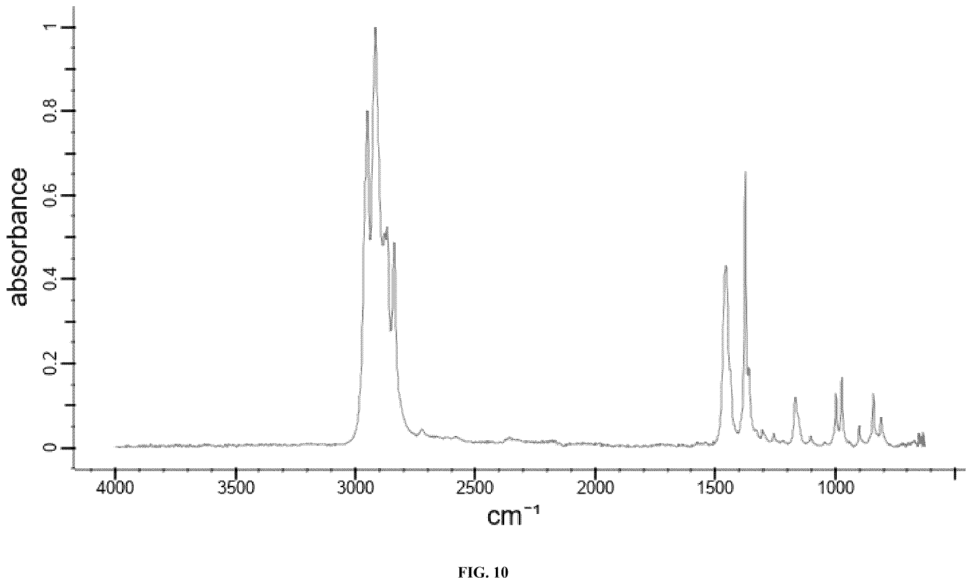

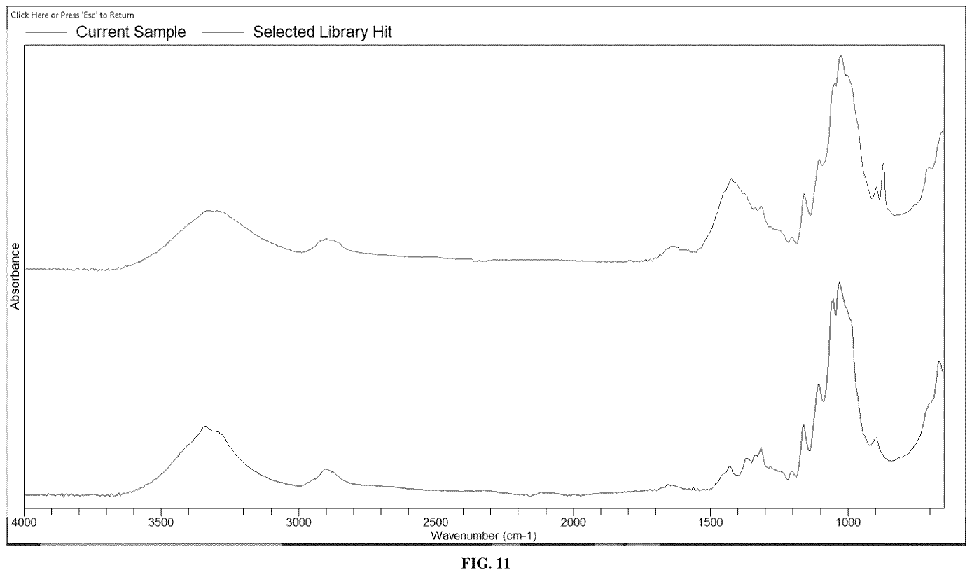

FIG. 9 shows the spectrum relating to the polyolefin fraction recovered. The spectrum is consistent with that of pure polypropylene of FIG. 10 (available on the SpectraBase® database of John Wiley & Sons, Inc., spectrabase.com/).spectra-database).

FIG. 11 shows the comparison between the spectra relating to the fraction of cellulose recovered (at the top) and the known one of the pure cellulose (below) (available on the SpectraBase® database of the John Wiley & Sons, Inc., spectrabase.com/). Spectra-Database).

The FIGS. 7-11 therefore demonstrate how the method according to the present invention is totally respectful of the chemical-physical properties of the treated materials allowing an efficient and quantitative recovery.

The contamination of the cellulose by SAP has been confirmed by the fact that its ability to absorb water increases: from 4.4 g of water per gram of pretreatment cellulose it goes up to 15.6 g of water per gram of post-treatment cellulose. The structural integrity of the 8.3 g of post-treatment SAP has been confirmed by the determination of its ability to absorb water: from 168 g of water per gram of pretreatment SAP, it goes up to 172 g of water by gram of post-treatment SAP. The values are consistent.

It can therefore, net of process losses, estimate a quantitative recovery of the intact SAP.

### Example 5

Verification of the stability of SAP to oxidative treatment in the conditions of known art (US 2022/0257823). Study case: Caroat® (Potassium peroxymonosulfate) with acidic pH.

A laboratory sample was prepared as follows: 22.1 g of pure cellulose+8.4 g of pure SAP+1.5 g of polyolefins+70.0 g of water. The sample constituted by dispersing SAP grains on the basis of cellulose and plastics, was sprinkled with water to mix it and to make it similar to the real material, on average humidity 65-70%. All the water is absorbed by the material. The 68.6% wet material was then treated with Caroat® at 8.5% by weight in acidic pH conditions in open reactor, in the conditions of known art (US 2022/0257823). To this purpose, a saturated solution of said acid oxidizer, i.e. the triple 2KHSO₅·KHSO₄·K₂SO₄ salt, has been sprayed on the material. The material was then placed in the autoclave and three cycles at 2.1 bar and 134° C. were performed. The sample extracted from the autoclave (ICAN—Clave® model) was ground mechanically, dried and subjected to mechanical separation, always by different metal mesh sieves covering the range included from 2 mm to 150 μm. The stacked sieves were placed on a vibro sifter (Retsch®), which made it possible to split the SAP grains, of dimensions below the millimeter, from cellulose flakes and plastic strings, larger than a millimeter. A minimum contamination of the SAP by small cellulose fibers is not excluded. A minimum contamination of the cellulose is not excluded by small grains of SAP remained trapped in the flakes. The three fractions in the measure of 21.8 g of cellulose probably partially contaminated by SAP, 4.1 g of SAP and 1.5 g of polyolefins, with a mass loss of 14.4%, higher than process losses estimated at 2%, have been recovered by mechanical separation. However, there still appears to be a clear shortage in the recovered SAP, net of losses and any residual contamination in cellulose. This loss is quantifiable at no less than 47% by weight.

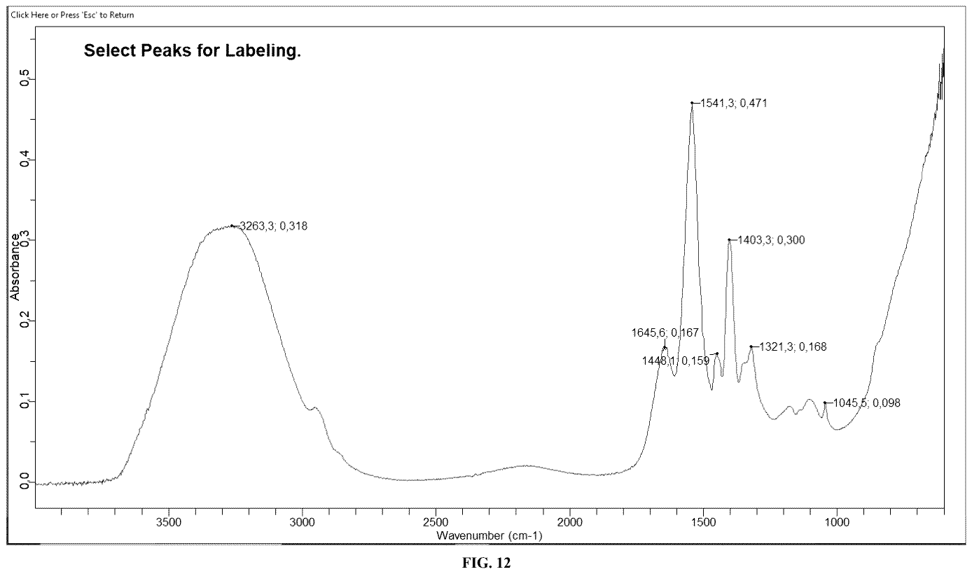

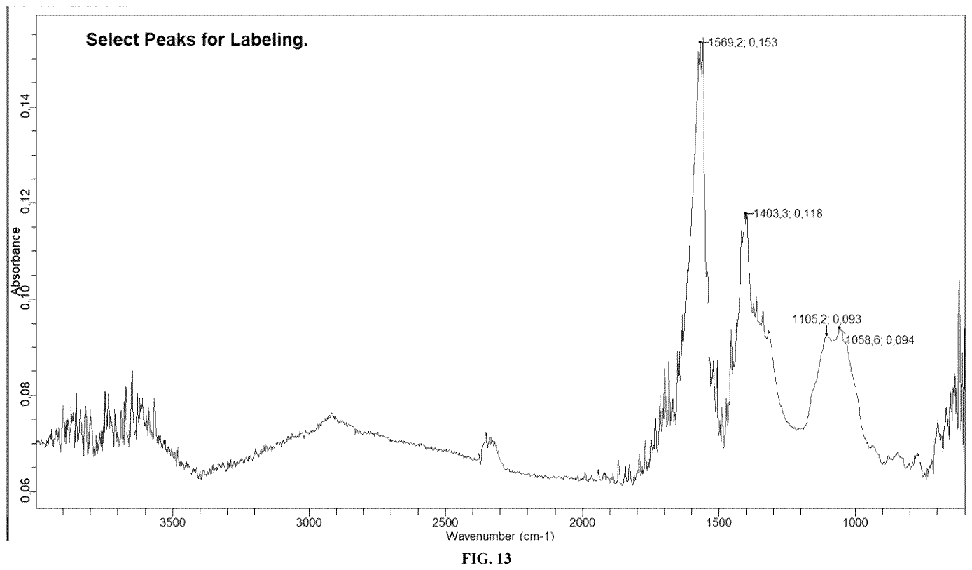

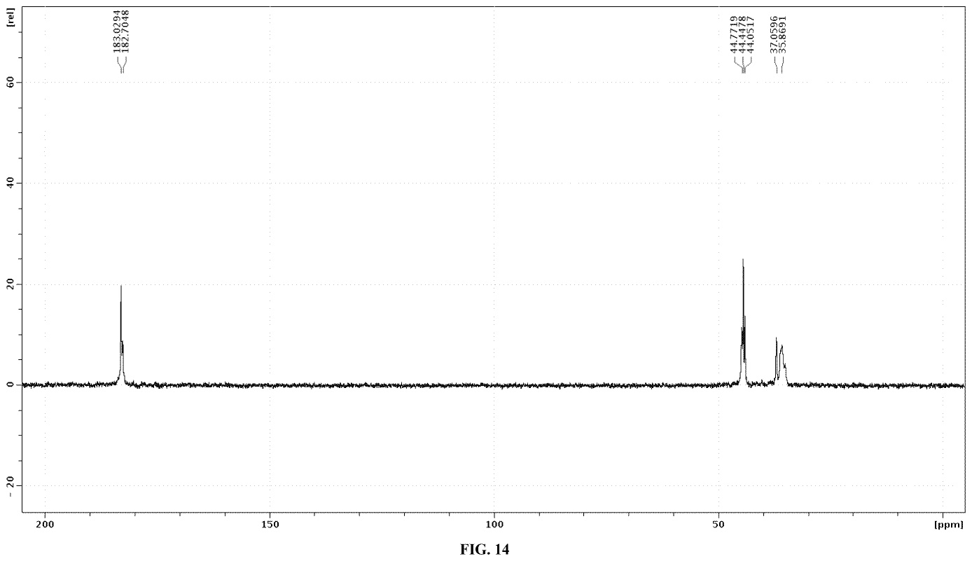

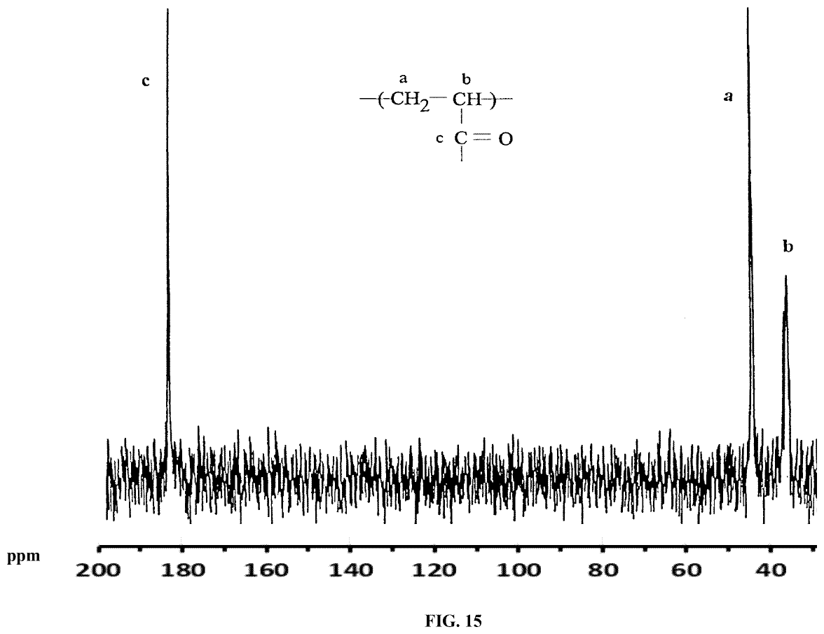

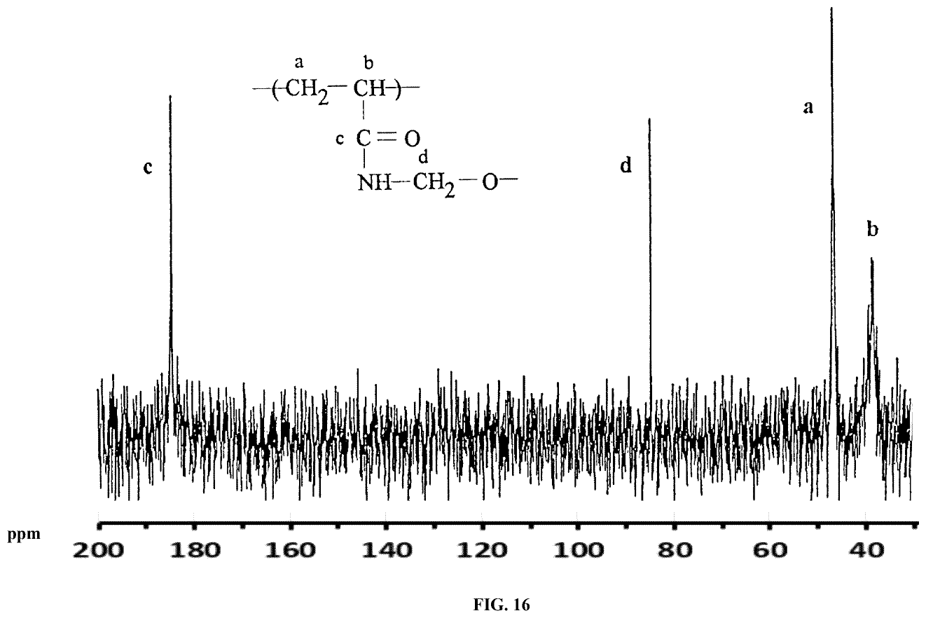

The identity of the three isolated fractions was confirmed by analysis performed by ATR and ¹³C-NMR spectrometry. The evident contamination of the cellulose from SAP has been confirmed by the fact that its ability to absorb water increases: from 4.4 g of water per gram of pre-treatment cellulose it goes up to 9.7 g of water per gram of post-treatment cellulose. It is not excluded that a contribution to the weight of the cellulose fraction is due to the presence of linear polyacrylate deriving from the oxidative dereticulation of SAP, which, soluble in the water, has imbued the cellulose fibers and has not increased its absorbent capacity. A release test performed on said cellulose releases a mixture in the water that presents at the ATR (Attenuated Total Reflectance) an IR spectrum (FIG. 13, at 1000-1100 cm⁻¹ there is the band of sulphates deriving from the Caroat®, instead, at about 2350 cm⁻¹ is outlined by the absorption peak of CO₂) compatible with pure LPA (FIG. 12 The 3000 cm⁻¹ band is due to water and is not characteristic of the product under examination). As a proof of this, the structural analysis performed in ¹³C-NMR carbon nuclear resonance spectrometry (FIG. 14) revealed the disappearance at 84 ppm of the signal relating to the methylenic bridges —CH₂— between heteroatoms, typical cross-linker (—NCH₂N—) of SAP (FIG. 16, spectrum in ¹³C-NMR of pure SAP [4]), so confirming its depolymerization. Not only, the ¹³C-NMR spectrum carried out on the SAP de-reticulated sample is superimposable with the spectrum of pure LPA (FIG. 15). [4]

Instead the structural integrity of the 4.1 g of SAP recovered post-treatment has been confirmed both by the IR spectrum and by the determination of its ability to absorb water: from 168 g of water per gram of pretreatment SAP, it goes up to 156 g of water for gram of post-treatment SAP. The values are consisting within the limits of the error of the method used, NFV Standard 19-002 of 1993-Determination of Absorbent Power.

It is therefore possible, net of process losses, to estimate only a semi-quantitative recovery of the intact SAP. The remaining part is LPA, linear polyacrylate, deriving from the oxidative dereticulation of SAP. This demonstrates how the use of particularly aggressive oxidizers (e.g., potassium peroxymonosulfate) in acid reaction conditions compromise the integrity of the super-absorbent polymers (SAP) by favoring an unwanted depolymerization in LPA.

### Example 6

### Determination of the Apparent Density Value of Only the Cellulosic Component of the Recovered Post-Treatment Fraction.

10 10.0 g samples of only cellulose fiber flakes deriving from a cellulose sample obtained after oxidative treatment were prepared. The residual plastic fraction has been evaluated/eliminated by selective extraction in xylene (UNIEN ISO 6427: 2013, Plastics: Determination of Matter Extractable by Organic Solvents), while the possible residual SAP fraction, which could compete negatively at the apparent density value, has been eliminated, by means of an aqueous step of the oxidative dereticulation of the super-absorbent polymer (WO 2021/130575 A1—a Method for separating and recovery super-absorbent polymers (SAP) from post-consumer absorbent sanitary products). The cellulose samples thus treated were dried in stove until constant weight, at (105±2°) C (humidity in materials: residue at 105° C., CNR IRSA method 2 notebook 64, volume 2, 1984; UNI EN 15414-3: 2011—Determination of the humidity content by drying method in stove). The cellulose flakes samples were then individually transferred to the 500 ml graduated container on a balance with a precision of 0.01 g, previously zeroed. The weight of the cellulose tested is noted, the balance is cleared, deionized water is added up to the 500.0 ml mark, the weight of the water added at room temperature is measured. By combining the two equations reported in the paragraph “Determination of the apparent density of the cellulose” in the “analytical methods” section of the present document, 10 apparent density values are obtained, from which the average value of (518±33) mg/ml is obtained with a relative uncertainty about the measure of 6%. The uncertainty arises in particular from the error relating to the reading of the volume corresponding to the 500 ml graduated container used.

### Example 7

### Determination of the Apparent Density Value of the Cellulose in the Entry Material to be Treated.

5 10.0 g samples of only cellulose fiber flakes deriving from a cellulose sample obtained from the padding of commercially available diapers, adult diapers and mattress protecting traverses, were prepared. The plastic fraction of these intact products has been easily removed. The SAP fraction has instead been eliminated by aqueous extraction, post oxidative dereticulation of the super-absorbent polymer (WO 2021/130575 A1—a method for separating and recovery super-absorbent polymers (SAP) from post-use absorbent sanitary products). Thus are dried up to constant weight about 66 g of pure cellulose obtained. 5 cellulose flakes samples of about 10 g were so individually transferred to a 500 ml graduated container on a balance with a precision of 0.01 g, and previously zeroed. The weight of the cellulose tested with the aforementioned precision is noted, the balance is cleared, deionized water is added up to the 500.0 ml mark, and the weight of the added water is measured. By combining the two equations reported in the paragraph “Determination of the apparent density of cellulose” in the “Analytical methods” section of the present document, 5 apparent density values are obtained, from which the average value of (720±32) mg/ml is obtained, with a relative uncertainty about the measure of 4%. The uncertainty arises mainly from the error relating to the reading of the volume corresponding to the 500 ml graduated container used. The value obtained is consistent with that obtained by similar measures conducted on samples of common “hydrophilic absorbent pure cotton wadding for sanitary use”, available on the market, for which the average value of (753±30) mg/ml has been obtained.

### Example 8

### Determination of the Specific Surface of the Pure Cellulose Contained in the Absorbent Sanitary Products to be Treated.

A known concentration solution of Methylene blue, 250.0 ml, 3.0 mM was prepared. From it are prepared 5 solutions diluted from 1.0 to 30.0 μM, to obtain the value of the molar extinction coefficient ε.

Seven masses of 2.0 g of pure cellulose derived from the padding of commercially available diapers, adult diapers and mattress protecting traverses, were placed in 7 containers and balanced for 24 hours with 100.0 ml of the Methylene blue solution with concentrations C from 2 to 300 μM. After 24 hours, the solutions separate from the cellulose, the V-volumes are determined, and the 664 nm absorbance values are read. Knowing the value of F and referring to formulas 8, 9, 10 reported in the paragraph “Determination of the specific surface of the cellulose fiber” in the “Analytical methods” section of the present document the corresponding concentrations C of the residual dye in solution are calculated, hence the values of Ni from equation (9). Nm is obtained from equation (8) and the specific surface value of 46 m²/g from equation (10).

### Example 9

Determination of the Specific Surface of the Cellulose Recovered after Performing the Process According to the Present Invention.

Seven masses of 2.0 g of recovered and separated after thermo-oxidative treatment with hypochlorite post-use cellulose, have been placed in 7 containers and balanced for 24 hours with 100.0 ml Methylene blue solution with concentrations Cᵢ from 2 to 300 μM. After 24 hours, the solutions are separated from the cellulose, the V volumes are determined, and the 664 nm absorbance values are read. Knowing the value of F and referring to formulas 8, 9, 10 reported in the paragraph “Determination of the specific surface of the cellulose fiber” in the “Analytical methods” section of the present document, the corresponding concentrations C of the residual dye in solution are calculated, from which the values of Ni from the equation (9). The value of Nm from the equation (8) and a specific surface value of 107 m²/g from equation (10) are obtained.

### REFERENCES

[1]C. Kaewprasit, E. Hequet, N. Abidi, J. P. Gourlot, Application of Methylene Blue Adsorption to Cotton Fiber Specific Surface Area Measurement: Part I. Methodology, Journal of Cotton Science, Vol. 2, pag. 164-173, 1998.
[2]L. Druel, T. Budtova, Aerogel-like (low density and high surface area) cellulose monoliths and beads obtained without supercritical- or freeze-drying, Cellulose, 30, 8339-8353, 2023.
[3] Gregg, S. J., and K. S. W. Sing. The physical adsorption of gases by nonporous solids: The type II isotherm. Eq. 2.1 pag. 41. 1982. In Adsorption, surface area and porosity. Academic Press, London.
[4] Liu, Z. S., Rempel, G. L., Preparation of SAP by crosslinking Acrylic Acid and Acrylamide Copolymers, Appl. Polym. Sci. 64,1345, 1997.

What is claimed is:

1. A method to sterilize and decontaminate post-use absorbent sanitary products contaminated with organic compounds deriving from body metabolism, wherein absorbent sanitary products comprise plastic fractions and at least one of first super-absorbent polymers (SAP) and first cellulose, the method comprises steps of:
grinding at least once the absorbent sanitary products to obtain grinded absorbent sanitary products, according to a homogeneous size equal to or lower than 40 cm;
sterilizing the grinded absorbent sanitary products by heating at a temperature equal to or lower than 140° C. and at a relative pressure between 1 bar and 3 bars to obtain sterilized absorbent sanitary products;
oxidizing the sterilized absorbent sanitary products with hypochlorite in an alkaline aqueous solution at a temperature lower than 80° C. to obtain oxidized sanitary products; and
inactivating the oxidized sanitary products with a reducing substance chosen from carboxylic organic acids, hydrogen peroxide, and sodium percarbonate.

2. The method according to claim 1, further comprising a separation step of materials obtained.

3. The method according to claim 1, wherein the grinding is performed during aspiration of an extractor hood, and an extracted flow is treated through a scrubber.

4. The method according to claim 1, wherein during an oxidation step, a concentration of hypochlorite ClO⁻, coming from sodium hypochlorite or calcium hypochlorite, is between 0.3 and 0.5 moles per kg of contaminated absorbent sanitary products to be treated [mol/kg]; and a volume of liquid is between 1 and 3 liters per kg of the contaminated absorbent sanitary products to be treated.

5. The method according to claim 1, wherein an inactivation of the oxidized sanitary products is performed at a pH higher than 7.

6. The method according to claim 5, wherein in the inactivation of the oxidized sanitary products, the reducing substance is the hydrogen peroxide.

7. The method according to claim 1, wherein the carboxylic organic acids are selected from the group consisting of citric acid, malic acid, tartaric acid, ascorbic acid, oxalic acid, and mixtures thereof.

8. The method according to claim 1, wherein in an inactivation step, an aliquot of a pH correcting compound is added, and the pH correcting compound is chosen from inorganic acids, keeping a pH higher than 7, wherein the inorganic acids comprise phosphoric acid, hydrochloric acid, sulfuric acid, or mixtures thereof.

9. The method according to claim 1, wherein a sterilization step is performed in an autoclave, and an oxidation step is performed in the same autoclave, wherein the autoclave is internally equipped with a layer resistant to oxidation and having non-sticking properties, wherein the layer comprised silicone or Teflon.

10. The method according to claim 1, wherein the post-use absorbent sanitary products comprise residues of drugs.

11. The method according to claim 1, wherein the post-use absorbent sanitary products are chosen from: diapers for babies, diapers for adults, absorbents for incontinence for adults, hygienic absorbents, bed traverse, sheets, covers, an absorbent material for bedding of cats comprising traverses and hygienic bags for animals, toilet paper, intimate hygiene wipes, and napkins.

12. A material, comprising, separated or separable from each other, aliquots of: plastics and at least one of second SAP and second cellulose, wherein the plastics and at least one of the second SAP and the second cellulose have substantially the same characteristics as the absorbent sanitary products before being subjected to the method according to claim 1, and the second cellulose has a specific surface higher than a specific surface of the first cellulose present in the absorbent sanitary products before being subjected to the method.

13. The material according to claim 12, wherein the second cellulose has the specific surface more than double the specific surface of the first cellulose present in the absorbent sanitary products before being subjected to the method.

14. The material according to claim 12, wherein the second cellulose has an apparent density lower than an apparent density of the first cellulose present in the absorbent sanitary products before being subjected to the method.

15. The method according to claim 2, wherein the grinding is performed during aspiration of an extractor hood, and an extracted flow is treated through a scrubber.

16. The method according to claim 2, wherein during an oxidation step, a concentration of hypochlorite ClO⁻, coming from sodium hypochlorite or calcium hypochlorite, is between 0.3 and 0.5 moles per kg of contaminated absorbent sanitary products to be treated [mol/kg]; and a volume of liquid is between 1 and 3 liters per kg of the contaminated absorbent sanitary products to be treated.

17. The method according to claim 3, wherein during an oxidation step, a concentration of hypochlorite ClO⁻, coming from sodium hypochlorite or calcium hypochlorite, is between 0.3 and 0.5 moles per kg of contaminated absorbent sanitary products to be treated [mol/kg]; and a volume of liquid is between 1 and 3 liters per kg of the contaminated absorbent sanitary products to be treated.

18. The method according to claim 2, wherein an inactivation of the oxidized sanitary products is performed at a pH higher than 7.

19. The method according to claim 3, wherein an inactivation of the oxidized sanitary products is performed at a pH higher than 7.

20. The method according to claim 4, wherein an inactivation of the oxidized sanitary products is performed at a pH higher than 7.
